(19) United States
(12) Patent Application Publication
(10) Pub. No.: US 2025/0188081 A1
(43) Pub. Date: Jun. 12, 2025

(54) Method for Production of Spiro-Oxindole and Indoxyl Derivatives of Mitragynine

(71) Applicant: Shaman Pharma, LLC, Kansas City, MO (US)

(72) Inventor: Patrick M. Kampmeyer, Collinsville, OK (US)

(21) Appl. No.: 18/974,352

(22) Filed: Dec. 9, 2024

Related U.S. Application Data
(60) Provisional application No. 63/607,872, filed on Dec. 8, 2023.

(51) Int. Cl. C07D 471/14; C07D 471/20

(52) U.S. Cl. CPC C07D 471/14; C07D 471/20

(57) ABSTRACT

A method for preparing a product compound of formula (II) includes purifying, from an extract, intermediate compound formula (I). The purification includes extracting mitragynine from a matrix using an extraction solvent to achieve an extract; basifying the extract, whereby the pH of the basified extract is in the range of 8-10; subjecting alkaloids precipitated in the extraction process to further extraction using an immiscible or partially miscible solvent; collecting an organic layer and the solvent from the further extraction to provide a residue concentrated in the intermediate compound formula (I); dissolving the residue in a reaction solvent to achieve an intermediate compound solution; and treating a mixture enriched with the intermediate compound solution with one or more reagents, whereby the treatment of the mixture results in the product compound of formula (II).

### CROSS-REFERENCE TO RELATED APPLICATIONS

This application claims the benefit of priority of U.S. Provisional Patent Application No. 63/607,872, filed Dec. 8, 2023, the disclosure of which is incorporated by reference herein in its entirety.

### FIELD OF THE INVENTION

The invention generally relates to a method for production of spiro-oxindole and indoxyl derivatives of mitragynine, and more particularly, to a method for pharmaceutical grade production of spiro-oxindole and indoxyl derivatives of mitragynine from 7-hydroxymitragynine.

### BACKGROUND

The chemical structures of the naturally occurring alkaloids found in M. Speciosa, with mitragynine (I) as a prime example, display a distinct 9-methoxy-5,6-dihydro-β-carboline chromophore, coupled with two essential side chains: an ethyl group at C20 and a methyl β-methoxy-acrylate moiety. These components constitute the fundamental structural framework of Corynanthe-type indole alkaloids. By subjecting these alkaloids to chemical oxidation, a diverse range of Corynanthe-type oxindoles can be generated which are of therapeutic interest. However, this process often yields a mixture of oxidation products due to competing oxidation reactions occurring at the nitrogen, C2, and C3 positions, potentially accompanied by rearrangements and over-oxidation.

Achieving the required chemo-selectivity and regio-selectivity demands entails not only the utilization of (a) site-selective oxidant(s) but also the presence of suitable substitutions at C2 and/or C3, in addition to a protective group on the nitrogen atom. Consequently, the limited pool of oxidants capable of orchestrating the oxidative rearrangement of tetrahydro-β-carbolines into spirooxindoles and 3-hydroxyindoles is not surprising. Among the oxidants previously documented, none exhibit optimal reactivity and selectivity concurrently, often leading to the formation of byproducts that are challenging to eliminate using conventional purification methods.

As of now, the synthesis of these compounds remains inefficient in terms of cost-effectiveness and environmental compatibility, while also failing to yield products that meet the standards for pharmaceutical acceptability.

### SUMMARY OF THE INVENTION

Existing literature has documented various oxidation methods for converting mitragynine into 7-hydroxymitragynine, such as the use of (Bis(trifluoroacetoxy)iodo)benzene (PIFA), monopersulfate (Oxone), and other oxidants. Additionally, previous studies have reported the conversion of 7-hydroxymitragynine to mitragynine pseudoindoxyl by employing alkoxide sources like sodium methoxide or sodium ethoxide. However, these approaches have encountered challenges stemming from poor selectivity and uncontrolled conversion, leading to the formation of unintended derivatives.

For instance, the highest reported conversion of mitragynine to 7-hydroxymitragynine, achieved using monopersulfate (Oxone), has reached a maximum of 51%. Similarly, the maximum documented yield of mitragynine pseudoindoxyl (II) from 7-hydroxymitragynine (I) through treatment with sodium methoxide stands at 31%. Both reactions result in the generation of byproducts that prove problematic to purify on a preparative scale.

In contrast, the methods introduced herein offer more controlled reactions under milder conditions through the utilization of co-oxidants, resulting in significantly fewer undesired byproducts. Moreover, for pharmaceutical preparations, conventional purification techniques like column chromatography become necessary, involving complex multiple steps that yield low product recoveries due to irreversible adsorption and decomposition on the solid support. The novel purification methodologies outlined below dramatically mitigate costs and resource consumption, simultaneously enhancing production efficiency and atom economy.

### Semi-Synthesis of 7-HydroxyMitragynine

The present invention relates to a process for preparation of a product compound of the formula (II).

The process involves purifying from an extract, the first intermediate compound of the formula (I).

In various embodiments, the present disclosure encompasses a method for producing the product compound of formula (II) (7-hydroxymitragynine) from M. Speciosa leaves or an extract thereof enriched in the compound of formula (I) (mitragynine). The method involves isolating the alkaloid fraction from the starting material via an additional pH-based extraction.

The starting material can encompass raw M. Speciosa leaves. Alternatively, it can involve an extract of M. Speciosa with varying mitragynine content. The mitragynine content can range from 10% to 50% by weight in certain embodiments, or from 50% to 100% by weight in other embodiments.

The pH-based extraction involves utilizing a water/alcohol (e.g., methanol or ethanol) mixture as the extraction solvent. The alcohol content can vary within the range of 0% to 90% by volume in some embodiments. Basification of the extract is achieved using agents such as ammonium hydroxide, sodium bicarbonate, sodium carbonate, potassium hydroxide, ammonium carbonate, diethylamine, sodium hydroxide, or mixtures thereof, aiming for a pH range of 8 to 10.

The precipitated alkaloids can then be extracted using an immiscible or partially miscible solvent, which may include dichloromethane (DCM), chloroform, ethyl acetate (EtOAc), diethyl ether, and others.

Subsequently, the enriched mixture of the initial intermediate compound undergoes treatment with an appropriate oxidant. This treatment is conducted under conditions effective in producing the product compound of formula (II).

The compound of formula (II) is then isolated from the reaction mixture using a chromatographic technique described herein.

### Semi-Synthesis of Mitragynine Pseudoindoxyl

Another aspect of the present invention relates to a process for preparation of a product compound of formula (III).

The process involves purifying from an extract or crude reaction mixture, the first intermediate compound of formula (II).

The purification of the initial intermediate can be effectively accomplished through a suitable liquid-liquid extraction method. In a particularly preferred embodiment, the liquid-liquid extraction system comprises a mixture of hexane, ethyl acetate, methanol, and water in specified proportions (2:7:1:9 v:v). Alternative extraction systems may involve different solvent combinations.

Subsequently, the procedure entails collecting the upper organic layer from this extraction and removing the solvent under reduced pressure. The residue is then redissolved in an appropriate reaction solvent or left to run in melt (solventless). In a preferred embodiment, the subsequent reaction takes place in melt.

Following this, the process entails subjecting a mixture that has been enriched in the compound represented by formula (II) to treatment with an appropriate reagent. This treatment is conducted to generate a compound characterized by the formula (III).

The compound of formula (III) is then isolated from the reaction mixture using a chromatographic technique described herein.

### Semi-Synthesis of Mitragynine φ-Pseudoindoxyl

Another aspect of the present invention relates to a process for preparation of a product compound of the formula (IV).

The process involves purifying from an extract, the first intermediate compound of the formula (I) using a pH-based extraction.

The pH-based extraction involves utilizing a water/alcohol (e.g., methanol or ethanol) mixture as the extraction solvent. The alcohol content can vary within the range of 0% to 90% by volume in some embodiments. Basification of the extract is achieved using agents such as ammonium hydroxide, sodium bicarbonate, sodium carbonate, potassium hydroxide, ammonium carbonate, diethylamine, sodium hydroxide, or mixtures thereof, aiming for a pH range of 8 to 10.

The precipitated alkaloids can then be extracted using an immiscible or partially miscible solvent, which may include dichloromethane (DCM), chloroform, ethyl acetate (EtOAc), diethyl ether, and others.

The subsequent step in the process entails treating a mixture that is enriched with the compound of formula (I) with appropriate reagents. This treatment is executed under conditions that are effective in generating the product compound characterized by the formula (IV).

The compound of formula (IV) is then isolated from the reaction mixture using a chromatographic technique described herein.

### Purification Using pH-Zone-Refining Centrifugal Partition Chromatography (CPC)

Another facet of the current innovation presents methodologies for achieving preparative-scale purification of products from the reaction crude mixture, utilizing pH-zone-refining Centrifugal Partition Chromatography (CPC). In one embodiment, a retainer is held in a liquid stationary phase through which a mobile phase flows, and the chromatographic process occurs between these two liquid phases. The separation process is conducted employing a two-phase solvent system, where the retainer is predominantly distributed within the stationary phase while the analytes are partitioned between the two phases. The column is initially filled with the stationary phase containing the retainer, followed by the injection of the sample. Subsequently, the mobile phase is eluted through the column. The mixture of reaction products is effectively resolved based on the differing affinities between the analytes and the retainer, as well as the eluter.

A significant advantage of the current method lies in the absence of a solid support within the column. Consequently, there is no requirement for immobilizing the retainer onto a solid stationary phase, which often entails a complex synthetic procedure. Moreover, earlier literature has documented instances of irreversible binding occurring on solid supports, accompanied by a notable degree of sample loss. These issues are circumvented through countercurrent liquid chromatography, as is the case with the present approach. Additionally, the liquid stationary phase is capable of accommodating a larger quantity of the retainer in comparison to the solid support stationary phase commonly found in conventional chromatographic columns. This novel technique offers a streamlined and efficient means of achieving preparative-scale separation, contributing to the optimization of product isolation from reaction mixtures.

According to one embodiment, a method for preparing a product compound of formula (II) includes purifying, from an extract, intermediate compound formula (I). The purification includes extracting mitragynine from a matrix using an extraction solvent to achieve an extract; basifying the extract, whereby the pH of the basified extract is in the range of 8-10; subjecting alkaloids precipitated in the extraction process to further extraction using an immiscible or partially miscible solvent; collecting an organic layer and the solvent from the further extraction to provide a residue concentrated in the intermediate compound formula (I); dissolving the residue in a reaction solvent to achieve an intermediate compound solution; and treating a mixture enriched with the intermediate compound solution with one or more reagents, whereby the treatment of the mixture results in the product compound of formula (II).

According to another embodiment, a method of preparing a product compound of formula (III) includes purifying, from an extract or crude reaction mixture, intermediate compound formula (II). The purification includes performing a liquid-liquid extraction, wherein the liquid-liquid extraction comprises 2 parts hexane, 7 parts ethyl acetate, 1 part methanol, and 9 parts water by volume; collecting an upper organic layer from the liquid-liquid extraction; removing solvent from the liquid-liquid extraction to produce a residue; dissolving the residue in a reaction solvent to produce a reaction solvent mixture; heating the reaction solvent mixture to 150-200° C.; and stirring the reaction solvent mixture for 5-60 minutes under inert atmosphere.

In still another embodiment, a method for preparing a product compound of formula (II) includes purifying intermediate compound formula (I). The purification includes extracting mitragynine from M. Speciosa leaves using an extraction solvent to achieve an extract; basifying the extract, whereby the pH of the basified extract is in the range of 8-10; subjecting alkaloids precipitated in the extraction process to further extraction using an immiscible or partially miscible solvent; collecting an organic layer and the solvent from the further extraction to provide a residue concentrated in the intermediate compound formula (I); dissolving the residue in a reaction solvent to achieve an intermediate compound solution; and treating a mixture enriched with the intermediate compound solution with one or more reagents, whereby the treatment of the mixture results in the product compound of formula (II).

### BRIEF DESCRIPTION OF THE DRAWINGS

FIG. 1 is the chemical compound for a Formula (I) Mitragynine.

FIG. 2 is the chemical compound for a Formula (II) 7-HydroxyMitragynine.

FIG. 3 is the chemical compound for a Formula (III) Mitragynine PseudoIndoxyl.

FIG. 4 is the chemical compound for a Formula (IV) Mitragyine φ-Pseudoindoxyl.

FIG. 5 is the chemical compound for an Intermediate Compound (I) Mitragynine.

FIG. 6 is the chemical compound for Intermediate Compound (II).

FIG. 7 is the chemical compound for a Byproduct 1.

FIG. 8 is the chemical compound for a Byproduct 2.

FIG. 9 is the chemical compound for a Byproduct 3.

FIG. 10 is the chemical compound for a Byproduct 4.

FIG. 11 is the chemical compound for a Byproduct 5.

FIG. 12 is the chemical compound for a Byproduct 6.

FIG. 13 is the chemical compound for a Byproduct 7.

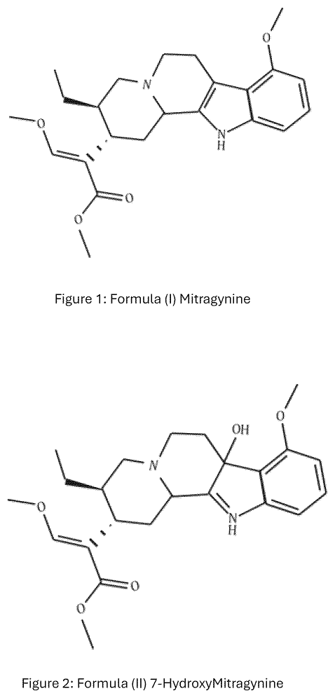

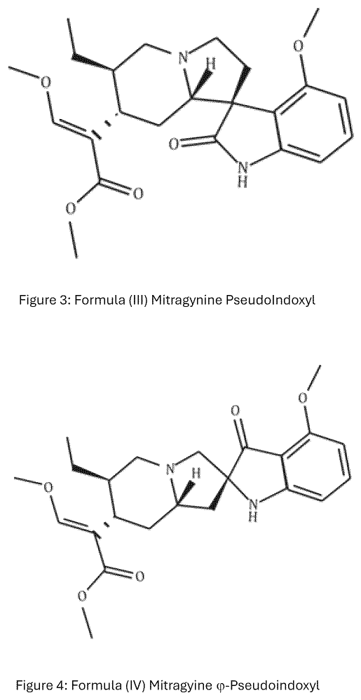

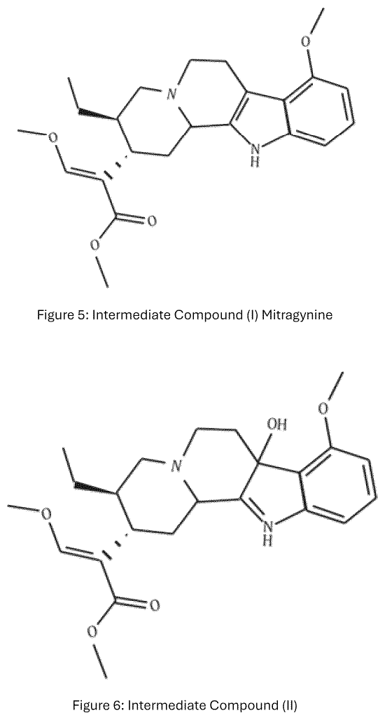

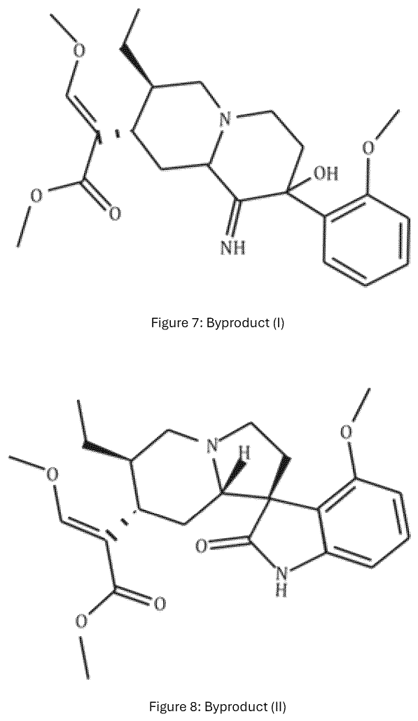

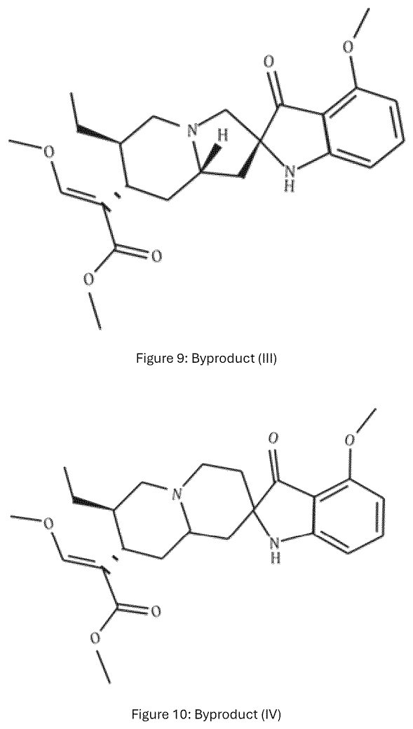

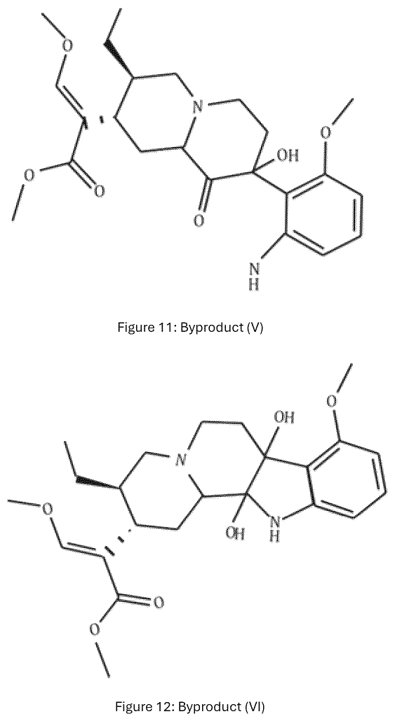

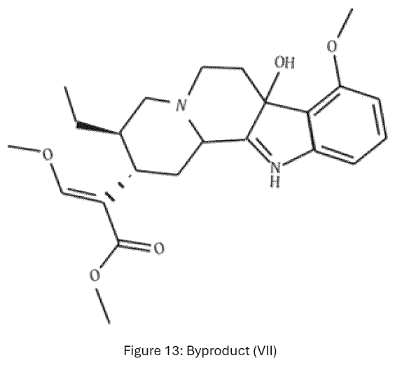

### DETAILED DESCRIPTION

### Definitions and Abbreviations

The following abbreviations are used herein:

HPLC, High performance liquid chromatography.

TLC, Thin layer chromatography.

As used herein, the term “retainer” is used to refer to a compound or substance that exhibits a high affinity for retaining or sequestering impurities, undesired components, or substances with lower pH values during the pH zone refining process. The retainer is an essential element in the disclosed method, as it selectively interacts with acidic or lower pH impurities, facilitating their removal from the target compound or material.

The retainer may possess specific functional groups or chemical properties that enable it to form reversible or irreversible interactions with acidic species present in the feedstock. These interactions may include ion-exchange processes, hydrogen bonding, coordination complexes, or any other suitable chemical affinity that promotes the preferential retention of low pH components while allowing the desired product or higher pH components to pass through the pH zone refining system.

The specific choice of the retainer will depend on the nature of the target compound or material to be purified, the impurities present, and the desired outcome of the pH zone refining process. The retainer should be carefully selected to ensure efficient and effective removal of impurities while minimizing any adverse effects on the final product's quality or purity.

As used herein, the term “eluter” refers to a compound or substance utilized in the pH zone refining process for Centrifugal Partition Chromatography (CPC). The eluter plays a crucial role in selectively releasing or eluting the target compound or desired components from the CPC column, following their separation and purification during the pH zone refining procedure.

In the CPC system, the eluter interacts with the stationary phase and the mobile phase, which contains the sample or feedstock to be purified. The eluter's chemical properties and affinity are carefully chosen to enable the targeted desorption of the purified compound or compounds of interest, while the rest of the components, including impurities and unwanted species, remain retained within the stationary phase.

The elution process may be achieved through various mechanisms, such as altering the mobile phase composition, adjusting pH, employing competing solvents, or changing the temperature. These factors influence the strength of the interaction between the eluter and the stationary phase, allowing for the efficient release of the target compound(s) from the column.

The selection of an appropriate eluter is critical to the success of the pH zone refining process in CPC. The eluter must possess high specificity for the target compound(s) and should not adversely impact the compound's purity or integrity during elution. Furthermore, the eluter should have sufficient compatibility with the CPC system to ensure smooth operation and reproducibility.

The eluter's design and implementation may vary based on the specific application of the pH zone refining in CPC, the properties of the target compound(s), and the complexity of the feedstock. Customized eluters can be tailored to optimize the purification process, enhancing the overall efficiency and yield of the CPC system.

As used herein, the term “separating” means to increase the amount of one component relative to the amounts of other components in a sample mixture. The mixture produced upon “separating” one component will be substantially free from the other components in the sample mixture, but may contain added quantities of solvents.

As used herein, the phrase “immiscible liquid phases” refers to liquids which may be partially miscible, but which separate into two phases having a liquid interface on standing. Typically, the two phases will comprise an organic phase and an aqueous phase. Suitable organic solvents include but are not limited to diethyl ether, hexane, ethyl acetate, methanol, methyl t-butyl ether, and acetonitrile.

In this context, the term “identifying” refers to the process of determining the presence of the desired compound in a specific sample or eluted fraction using spectroscopic techniques, such as UV detection, refractive index detection, mass spectroscopy, and IR detection. Additionally, compounds can be “identified” by comparing their elution times using High-Performance Liquid Chromatography (HPLC).

### Semi-Synthesis of 7-HydroxyMitragynine

The invention relates to a process for preparation of a product compound of the formula (II).

The process involves purifying from an extract, the first intermediate compound of the formula (I).

The pH-based extraction involves utilizing a water/alcohol (e.g., methanol or ethanol) mixture as the extraction solvent. The alcohol content can vary within the range of 0% to 90% by volume in some embodiments. Basification of the extract is achieved using agents such as, for example, ammonium hydroxide, sodium bicarbonate, sodium carbonate, potassium hydroxide, ammonium carbonate, diethylamine, sodium hydroxide, or mixtures thereof, aiming for a pH range of 8 to 10.

Subsequently, the alkaloids that precipitate from this pH-based extraction process are subjected to extraction using an immiscible or partially miscible solvent. This selection of solvents encompasses dichloromethane (DCM), chloroform, ethyl acetate (EtOAc), diethyl ether, and similar options.

The organic layer is then collected and the solvent removed under reduced pressure to afford a residue concentrated in the compound of formula (I). The residue is then dissolved in a suitable reaction solvent. In a preferred embodiment, the reaction solvent is water/methanol in a ratio ranging from 1:5 to 5:1. In another embodiment, the reaction solvent is water/ethanol or water/acetone in similar ratios. The process then involves treating a mixture enriched with the first intermediate compound using the appropriate reagents. This treatment takes place under conditions that efficiently culminate in the generation of the product compound as defined by formula (II).

The method of the invention employs a mild and selective oxidizing agent for this purpose. Examples of such agents include PDC (pyridinium chlorochromate), TPAP (tetrapropylammonium perruthenate), or combinations thereof. Additionally, the co-oxidant employed in this method could be AcOH (acetic acid), TEMPO (2,2,6,6-tetramethylpiperidine-1-oxyl), or combinations thereof.

An alternative methodology involves the utilization of oxidants in the form of metals, such as silver oxide (Ag2O), coupled with an appropriate ligand. For instance, Ag2O can be used alongside N-methylmorpholine N-oxide (NMO), along with a ligand such as PPh3 (triphenylphosphine) or DPPP (1,3-bis (diphenylphosphino) propane).

Furthermore, other oxidants suitable for this method encompass 2-iodobenzoic acid, potassium permanganate, hydroxy{[(4-methylphenyl)sulfonyl]oxyl phenyliodide (HTIB), Dess-Martin Periodinane, diacetoxyiodobenzene, iodobenzene, mCPBA, N-bromosuccinimide, OsO4, CrO3, t-BuOCl, DCC, Iron (III) chloride, selenium dioxide, Hydroxylamine-O-sulfonic acid, N-hydroxypthalimide, or combinations thereof.

Another embodiment of the invention pertains to the hydroxylation of compound (I) to produce a product of formula (II) in the presence of a transition metal catalyst or catalytic amount of acid. This hydroxylation process is achieved through the utilization of O2 (Oxygen) or H2O2 (hydrogen peroxide), individually or in combination, in the presence of either a transition metal catalyst or an acid (such as HCl or HBr).

Subsequently, the resultant product is quenched with water and extracted using an immiscible solvent such as DCM. The solvent is then removed under reduced pressure, yielding the reaction crude. This crude mixture is further dissolved in water and a miscible solvent, commonly an alcohol (e.g., ethanol or methanol). The mixture undergoes a basification process using ammonium hydroxide or another appropriate base. This procedure results in the generation of a crude mixture that is enriched in compound (II).

### Semi-Synthesis of Mitragynine Pseudoindoxyl

Another embodiment relates to a process for preparation of a product compound of the formula (III).

The process involves purifying from an extract or crude reaction mixture, the first intermediate compound of the formula (II).

Purification of the second intermediate is accomplished through a suitable liquid-liquid extraction method. In a preferred embodiment, the extraction system comprises hexane, ethyl acetate, methanol, and water in specified proportions (2:7:1:9 v:v). Subsequent steps involve collecting the upper organic layer, solvent removal, and redissolving the residue in an appropriate reaction solvent. Notably, in a favored embodiment, the ensuing reaction proceeds devoid of a solvent.

The ensuing procedure entails elevating the temperature of the resulting mixture to a range of 150° C. to 200° C., either without a solvent or in a suitable solvent. This mixture is then stirred for a duration of 5 to 60 minutes under an inert atmosphere (nitrogen, argon, etc.), leading to the formation of compound with formula (III).

Another embodiment involves the hydroxylation and oxidative rearrangement of compound (I) to form the compound of formula (III), facilitated by green oxidation halide catalysis. In one embodiment, compound (I) undergoes treatment with an appropriate oxidant, complemented by a catalytic quantity of a halogen source. The potential oxidants encompass H2O2, Oxone, O2, or hypervalent iodine species such as bis (trifluoroacetoxy) iodobenzene (PIFA), diacetoxyiodobenzene, iodobenzene (PIDA), or any of the previously mentioned hypervalent iodine species, individually or in combination. Correspondingly, the halogen source in some instances consists of halogen diatom (e.g., Cl2, Br2) or compounds like HX (e.g., HBr, HCl) or KX (e.g., KBr, KCl).

### Semi-Synthesis of Mitragynine φ-Pseudoindoxyl

Another embodiment relates to a process for preparation of a product compound of the formula (IV).

The process involves purifying from an extract, the first intermediate compound of the formula (I).

In various embodiments, the extract's purity can range from 10% to 100%. The pH-based extraction involves utilizing a water/alcohol (e.g., methanol or ethanol) mixture as the extraction solvent. The alcohol content can vary within the range of 0% to 90% by volume in some embodiments. Basification of the extract is achieved using agents such as ammonium hydroxide, sodium bicarbonate, sodium carbonate, potassium hydroxide, ammonium carbonate, diethylamine, sodium hydroxide, or mixtures thereof, aiming for a pH range of 8 to 10.

Subsequently, the alkaloids that precipitate from this pH-based extraction process are subjected to extraction using an immiscible or partially miscible solvent. This selection of solvents encompasses dichloromethane (DCM), chloroform, ethyl acetate (EtOAc), and similar options.

The organic layer is then collected and the solvent removed under reduced pressure to afford a residue concentrated in the compound of formula (I). The residue is then dissolved in a suitable reaction solvent. In a preferred embodiment, the reaction solvent is water/methanol in a ratio ranging from 1:5 to 5:1. In another embodiment, the reaction solvent is water/ethanol or water/acetone in similar ratios. The process then involves treating a mixture enriched with the first intermediate compound using an appropriate oxidant. This treatment takes place under conditions that efficiently culminate in the generation of the product compound as defined by formula (II).

The treatment step can take place within an organic solvent. In a particular embodiment, the solvent is aprotic. Examples of organic solvents comprise hexane, heptane, toluene, xylene, dichloromethane, and mixtures thereof. The treatment can be executed at temperatures ranging from about −20° C. to about 100° C. Alternatively, in a different embodiment, the treatment can occur within a temperature range of about −20° C. to about 50° C.

In one specific embodiment, the transformation of compound (I) to compound (IV) is realized through hydroxylation and oxidative rearrangement of compound (I) using halide catalysis. In an alternate embodiment, compound (I) undergoes treatment with an appropriate oxidant alongside a catalytic quantity of a halogen source. This oxidant is H2O2 in one embodiment, Oxone in another embodiment, O2 in another, or hypervalent iodine species such as bis(trifluoroacetoxy)iodobenzene (PIFA), diacetoxyiodobenzene (PIDA), iodobenzene, or mixtures thereof in yet another embodiment. The halogen source could include halogen diatom (e.g., Cl2, Br2) or compounds like HX (e.g., HBr, HCl) or KX (e.g., KBr, KCl) in specific instances.

### Purification Using pH-Zone-Refining Centrifugal Partition Chromatography (CPC)

The utilization of oxidants in previous approaches to generate compounds (II) and (III) has encountered difficulties primarily concerning selectivity. The resultant reaction mixtures in their crude form encompass a significant multitude of impurities. Employing traditional techniques such as column chromatography for isolating compounds (II), (III), and (IV) necessitates intricate multi-step processes, ultimately yielding low product recoveries due to irreversible adsorption and decomposition on the solid support.

The ensuing list outlines potential byproducts that may arise during the conversion from compound (I) to compound (II) and from compound (II) to compound (III). These byproducts are effectively eliminated from the products by means of the methodologies described herein.

In the context of any approach outlined herein, it is feasible to utilize any blend of solvents capable of separating into two distinct phases when allowed to settle. These phases can individually consist of organic solutions or aqueous solutions. A favored embodiment involves one phase containing one or more organic solvents, while the other phase is primarily aqueous. The solvents of preference are those that, upon phase separation, exhibit a density difference of at least 0.05 g/mL between the two phases.

The alignment of phases in relation to each other can occur either before or during the chromatography process. In methods utilizing an acidified or basified liquid phase or phases, the equilibrium between the two phases might be established before subjecting them to acidification or basification individually. In scenarios where a basic aqueous phase functions as a mobile phase for the separation of acidic solutes, phase equilibration can be initiated after the aqueous phase has been rendered basic. Conversely, if an acidic aqueous phase is utilized as the mobile phase for the separation of basic solutes, the phase equilibration could take place subsequent to the aqueous phase's acidification.

A preferable approach involves equilibrating the phases in their neutral states by blending them together and subsequently allowing them to separate before introducing the stationary phase into the column. In cases where the phases are equilibrated in their neutral forms, the chromatography column's stationary phase can be acidified (for the separation of acidic solutes) or basified (for the separation of basic solutes) prior to its introduction.

The solvent system for pH zone refining Centrifugal Partition Chromatography (CPC) is chosen primarily based on the partition coefficients (K) between the two phases, encompassing the eluter, retainer, and the target analyte. The preferable configuration involves the retainer predominantly distributed within the stationary phase, while the eluter occupies the mobile phase. Simultaneously, the analytes should possess K values ranging from about 0.3 to 1.0, ensuring a relatively uniform distribution between the two phases.

In a favored embodiment, the solvent system consists of a combination of hexane, ethyl acetate, methanol, and water. More specifically, the preferred solvent system includes hexane, ethyl acetate, methanol, and 10 mM HCl.

The precise degree of acidity and basicity within the two phases is not of primary significance. Generally, the most favorable outcomes are attained by employing an acidic phase with a pH exceeding approximately 4, preferably below approximately 3. Correspondingly, for the basic phase, the optimal pH ranges around 8, with a preference for values below 9 in most instances. Incorporating a more basic mobile phase leads to shorter elution times for acidic compounds, while a more acidic mobile phase decreases the elution times of basic samples.

Within a subset of embodiments, the method presented in this invention finds application in the segregation of reaction mixture products within a sample mixture utilizing CPC. This involves establishing equilibrium between two solvent phases that do not readily mix, resulting in a two-phase amalgam. Subsequently, a CPC column is loaded with the initial liquid phase of the mixture, containing the retainer. This liquid phase incorporates both the eluter and the sample. Consequently, fractions housing different constituents of the mixture are expelled, gathered, and characterized.

Each liquid phase can be classified as either an organic phase or an aqueous phase, functioning independently. In a preferred configuration, the initial liquid phase takes the form of an organic phase, while the subsequent liquid phase is aqueous. Furthermore, in an even more favored arrangement, the initial liquid phase is organic, while the succeeding liquid phase is characterized as an acidic aqueous phase.

In a preferred embodiment, the first liquid phase is made acidic by incorporating an organic acid, namely acetic acid, trifluoroacetic acid, propionic acid, or butanoic acid and the second liquid phase is rendered basic using either ammonia or NaOH.

### Preparation of Solvent Phases and Sample Solutions

The solvent pair was prepared through the following procedure: hexane, ethyl acetate, methanol, and distilled water (or a diluted HCl solution) are combined in a separatory funnel at room temperature, after which the two phases are separated. The upper organic phase, containing the retainer, is designated as the stationary phase, while the lower aqueous phase is designated as the mobile phase.

To formulate the sample solutions, 15 g of the crude reaction product is dissolved in 300 ml of the upper phase (with 10 mM TEA) from the two-phase solvent system, at a ratio of 1:20 (m/v). The resulting mixture is subjected to two extractions with 300 ml of the lower phase (with 10 mM TEA), discarding the lower phase after each extraction. Subsequently, the upper phase underwent two extractions with 300 ml of the lower phase (with 10 mM HCL). The combined acidified lower phases are made basic by adding 25-28% NH4OH to achieve a pH of 10. This solution is then evaporated to dryness under reduced pressure using a rotary evaporator. The resulting residue is dissolved in water at a ratio of 1:10 (m/v) and subjected to two extractions with ethyl acetate in a 1:1 (v/v) ratio. The ethyl acetate extract is collected and evaporated to dryness, yielding 4.0 g of refined alkaloids. From this, a portion of 2.5 g of the refined alkaloids is employed for pH-zone-refining CPC separation.

### Separation Procedure

The pH-zone-refining CPC separation procedure employs a two-phase solvent system consisting of n-hexane-ethyl acetate-methanol-water (3:7:1:9, v/v). The upper phase, containing 10 mM TEA, serves as the stationary phase, while the lower phase, containing 10 mM HCl, functions as the mobile phase.

The sample solution is created by dissolving a specific quantity of either crude or refined alkaloids in a mixture of 10 ml of basified upper phase and 10 ml of the lower phase with HCl removed.

To initiate the process, the column is initially filled with the stationary phase. Subsequently, the aforementioned prepared sample is injected via the sample injection valve. The rotor is then rotated at 850 rpm, and the mobile phase is pumped through the column in a head-to-tail direction at a flow rate of 1.5 ml/min. The temperature is maintained at 25° C. Detection of the effluent is conducted at a wavelength of 254 nm, and fractions of 5 ml are manually collected into test tubes. The pH of each eluted fraction is determined using a pH meter, while the purity was assessed using High-Performance Liquid Chromatography (HPLC).

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention. Further, it will be understood that certain features and subcombinations may be of utility and may be employed within the scope of the disclosure. Further, various steps set forth herein may be carried out in orders that differ from those set forth herein without departing from the scope of the present methods. This description shall not be restricted to the above embodiments.

It is to be understood that while certain forms of the present invention have been illustrated and described herein, it is not to be limited to the specific forms or arrangement of parts described and shown.

What is claimed is:

1. A method for preparing a product compound of formula (II), comprising:
purifying, from an extract, intermediate compound formula (I), comprising the steps of:
extracting mitragynine from a matrix using an extraction solvent to achieve an extract;
basifying the extract, whereby the pH of the basified extract is in the range of 8-10;
subjecting alkaloids precipitated in the extraction process to further extraction using an immiscible or partially miscible solvent;
collecting an organic layer and the solvent from the further extraction to provide a residue concentrated in the intermediate compound formula (I);
dissolving the residue in a reaction solvent to achieve an intermediate compound solution;
treating a mixture enriched with the intermediate compound solution with one or more reagents, whereby the treatment of the mixture results in the product compound of formula (II).

2. The method of claim 1, wherein the extract solvent comprises a water/alcohol mixture, and wherein the alcohol content is within the range of 0-90% by volume.

3. The method of claim 2, wherein the basifying step utilizes an agent selected from the list consisting of ammonium hydroxide, sodium bicarbonate, sodium carbonate, potassium hydroxide, ammonium carbonate, diethylamine, sodium hydroxide, and mixtures thereof.

4. The method of claim 3, wherein the solvent for the further extraction is selected from the list consisting of dichloromethane, chloroform, ethyl acetate, and diethyl ether.

5. The method of claim 4, wherein the collection step is performed under pressure.

6. The method of claim 5, wherein the reaction solvent is water/methanol or water/acetone.

7. The method of claim 6, wherein the ration of water to methanol or acetone ranges from 1:5 to 5:1.

8. The method of claim 7, further including utilizing an oxidizing agent, wherein the oxidizing agent is selected from the list consisting of pyridinium chlorochromate, tetrapropylammonium perruthenate, and combinations thereof.

9. The method of claim 8, further comprising utilizing a co-oxidant selected from the list consisting of acetic acid; 2,2,6,6-tetramethylpiperidine-1-oxyl, and combinations thereof.

10. The method of claim 1, wherein the basifying step utilizes an agent selected from the list consisting of ammonium hydroxide, sodium bicarbonate, sodium carbonate, potassium hydroxide, ammonium carbonate, diethylamine, sodium hydroxide, and mixtures thereof.

11. The method of claim 1, wherein the solvent for the further extraction is selected from the list consisting of dichloromethane, chloroform, ethyl acetate, and diethyl ether.

12. The method of claim 1, wherein the collection step is performed under pressure.

13. The method of claim 1, wherein the reaction solvent is water/methanol or water/acetone.

14. The method of claim 13, wherein the ration of water to methanol or acetone ranges from 1:5 to 5:1.

15. The method of claim 1, further including utilizing an oxidizing agent, wherein the oxidizing agent is selected from the list consisting of pyridinium chlorochromate, tetrapropylammonium perruthenate, and combinations thereof.

16. The method of claim 15, further comprising utilizing a co-oxidant selected from the list consisting of acetic acid; 2,2,6,6-tetramethylpiperidine-1-oxyl, and combinations thereof.

17. The method of claim 1, further including utilizing an oxidizing agent in the form of a metal coupled with an appropriate ligand.

18. The method of claim 1, further including utilizing an oxidizing agent selected from the list consisting of 2-iodobenzoic acid, potassium permanganate, hydroxy{[(4-methylphenyl)sulfonyl]oxyl} phenyliodide (HTIB), Dess-Martin Periodinane, diacetoxyiodobenzene, iodobenzene, mCPBA, N-bromosuccinimide, OsO4, CrO3, t-BuOCl, DCC, Iron (III) chloride, selenium dioxide, Hydroxylamine-O-sulfonic acid, N-hydroxypthalimide, or combinations thereof.

19. A method of preparing a product compound of formula (III), comprising:
purifying, from an extract or crude reaction mixture, intermediate compound formula (II), comprising the steps of:
performing a liquid-liquid extraction, wherein the liquid-liquid extraction comprises 2 parts hexane, 7 parts ethyl acetate, 1 part methanol, and 9 parts water by volume;
collecting an upper organic layer from the liquid-liquid extraction;
removing solvent from the liquid-liquid extraction to produce a residue;
dissolving the residue in a reaction solvent to produce a reaction solvent mixture;
heating the reaction solvent mixture to 150-200° C.; and
stirring the reaction solvent mixture for 5-60 minutes under inert atmosphere.

20. A method for preparing a product compound of formula (II), comprising:
purifying intermediate compound formula (I), comprising the steps of:
extracting mitragynine from M. Speciosa leaves using an extraction solvent to achieve an extract;
basifying the extract, whereby the pH of the basified extract is in the range of 8-10;
subjecting alkaloids precipitated in the extraction process to further extraction using an immiscible or partially miscible solvent;
collecting an organic layer and the solvent from the further extraction to provide a residue concentrated in the intermediate compound formula (I);
dissolving the residue in a reaction solvent to achieve an intermediate compound solution;
treating a mixture enriched with the intermediate compound solution with one or more reagents, whereby the treatment of the mixture results in the product compound of formula (II).
